(19) United States
(12) Patent Application Publication
(10) Pub. No.: US 2003/0022193 A1
(43) Pub. Date: Jan. 30, 2003

(54) Method for purification and subsequent determination of double-strand DNA

(76) Inventors: Wilhelm Pluster, Hamburg (DE); Peter Kunze, Glen Cove, NY (US); Thomas Kolzau, Hamburg (DE)

Correspondence Address: DAVID TOREN, ESQ., SIDLEY, AUSTIN, BROWN & WOOD, LLP, 787 SEVENTH AVENUE, NEW YORK, NY, 10019-6018, US

(21) Appl. No.: 10/060,465

(22) Filed: Jan. 30, 2002

(30) Foreign Application Priority Data
Jan. 31, 2001 (DE) 101 04 025.3

(51) Int. Cl. C12Q 1/68; C12P 19/34

(52) U.S. Cl. 435/6; 435/91.2

(57) ABSTRACT

A method for the purification and subsequent determination of double-strand DNA in which the DNA is immobilized on a solid phase, wherein during purification the DNA is immobilized on a probe using a residue having an affinity for DNA, whereby the probe itself is bound to a solid phase or is coupled in a later step to a solid phase and the solid phase is the surface of a reaction vessel, a chip or a tip and subsequent further purification for purposes of determination of the DNA is initiated directly using the nucleic acids immobilized on the solid phase.

### BACKGROUND

Conventional preparation protocols for double-strand DNA, particularly for plasmid DNA, are comprised of several process steps. Initially, the cells from which the DNA is to be isolated are concentrated, for example by centrifugation. The resuspended cell pellet is then subjected to a multi-step lysis (for example, alkaline lysis). The lysate is then again centrifuged in order to remove any cellular debris or similar material.

“All-in-one” methods for purification and determination of mRNA are well-known in the prior art. In the method disclosed in WO 99/32654 or EP 0754764, purification is based on a hybridization of the mRNA single strand on a surface modified using oligo-dT probes. As taught in the invention, however, this is not possible in the purification of double-strand DNA. To that extent the known methods remain limited to purification of mRNA.

### SUMMARY OF THE INVENTORY

In the method according to the invention, during purification the DNA is immobilized on a probe using a residue having an affinity for DNA. The probe itself is already immobilized on a solid phase or is immobilized on a solid phase in a further step, said solid phase being the surface of a reaction vessel, a chip or a tip.

The method according to the invention provides that the double-strand DNA on the modified solid phase can then be further processed for the purpose of its determination. Thus, the necessity of changing vessels or transferring the isolated DNA is eliminated.

The invention thus provides a particularly convenient method for purification and determination of double-strand DNA. This type of “all-in-one method” requires minimum pipetting and other process steps. In the simplest case, the sample from which the double-strand DNA is to be obtained, is pipetted into a reaction vessel that has been modified in accordance with the invention. After a period of incubation, the sample liquid is removed from the reaction vessel, whereby at least a portion of the double-strand DNA originally present in the sample is isolated/immobilized on the wall of the vessel. In the method according to the invention the double-strand DNA immobilized on the solid-phase carrier is presented in such a manner that a hybridization reaction can proceed immediately.

The DNA isolated according to the invention in a reaction vessel, can be easily amplified in a further processing step, for example, by means of PCR and then determined. Also conceivable within the scope of the invention, is processing using the Cycle Sequencing Principle. It is even further possible to detect the bound DNA without prior amplification directly by using, for example, appropriate probes.

If further processing by means of PCR is done, then the reaction vessels, chips, or tips modified according to the invention must have the appropriate thermal stability.

The probes bound to the surface of the solid-phase carrier used for DNA isolation need not necessarily be heat-stabile or form permanent complexes with the DNA and/or the surface. Durable, for example, complexes that survive a PCR are only then necessary if the reaction vessel is to be archived together with the reaction vessel for documentation purposes (for example, in order to re-do a PCR at a later time). In principle, it is not even necessary that the DNA, after isolation and initiation of the further processing, remain immobilized on the solid-phase to the end of said processing.

The inventive probes themselves can have various residues with affinity for double-strand DNA. One possibility is to provide the probes with so-called zinc fingers that bind to specific sections (minor grooves) of double-strand DNA. For further details, reference is made to the publication by Rhodes and Klug in Scientific American, 1993, p. 53 in which zinc fingers are described that are suitable according to the invention.

Other compounds that bind specifically to certain regions of the double-strand DNA, the so-called “sequence seekers”, can be used. These are selective polyamides that bind to specific nucleotide sequences of the double-strand DNA. For more specific information, reference is made to WO 98/37067 and WO 98/49142 that disclose a number of polyamides compatible with the invention.

Further, triple helix forming compounds can be used on the probes as the affinity residues. These probes, too, bind at specific sequence regions of the double-strand DNA to be isolated by anchoring an additional, third complementary strand. For additional information in this context, reference is made, for example, to U.S. Pat. No. 5,401,632 and U.S. Pat. No. 5,482,836.

The affinity residues described in the foregoing bind specifically to specific sequence segments of the double-strand DNA or to regions with a specific conformity.

In a further variant of the invention, it is also possible to provide residues that bind non-specifically to the double-strand DNA. In this connection, for example, a compound can be used as the affinity residue that provides the ionic coupling sites to which double-strand DNA can non-specifically anchor; that is, with sections of any sequence.

According to the invention and as is already well-known in the prior art, probes are used that are synthesized directly on the surface of the reaction vessels. It is also conceivable that reaction vessels, chips or tips are used, to which the probes are bound either before DNA processing or in the isolation step. A coupling site can be provided between the probe and the surface of the reaction vessel or the chip; for example, coupling using biotin-streptavidin. Further conceivable also are other groups that can be mutually coupled and, on the one hand provided on the probe and on the other hand on the surface of the reaction vessel. The advantage of such subsequent coupling of the probe to the surface is that different probes or similar can be coupled to the surface as needed, depending on for which application the reaction vessels are intended; thus, universal reaction vessels are possible.

### DETAILED DESCRIPTION OF THE PREFERRED EMBODIMENT

Purification and Amplification of Ironically Immobilized gDNA

Raw lysates of rat liver are used as the sample material. The lysates were transferred into Eppendorf centrifuge tubes whose surfaces were chemically modified using appropriate ionic coupling sites to which the double-strand DNA can non-specifically anchor.

Incubation followed for 10 to 20 minutes in the presence of various buffers.

| A | | | B | | |
|---|---|---|---|---|---|
| 10 | mM | Tris/HCL (pH 8.0) | 100 | mM | NaCl |
| 0.1 | M | EDTA | 10 | mM | Tris/HCL (pH 8.0) |
| 20 | μg/ | RNAse A | 0.1 | M | EDTA |
| | mL | | | | |
| 0.5 | % | SDS | 0.1 | mg/ml | Proteinase K |
| | | | 0.5 | % | SDS |
| C | | | | | |
| 10 | mM | Tris/HCL (pH 8.0) | | | |
| 0.1 | mM | EDTA | | | |
| 150 | mM | NaCl | | | |
| 0.5 | % | NP 40 | | | |

After incubation, the lysate was removed and the vessel washed. Then one fragment of the GAPDH-gene at a time was amplified in the vessels using PCR.

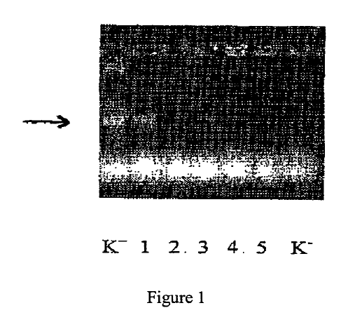

Aliquots of the PCR preparations were then electrophoretically analyzed in a 1% agarose gel. The result is shown in FIG. 1 which depicts an agarose gel with seven differently charged tracks (K⁺, 1-5, K⁻): (K⁺) control with commercially obtainable gDNA, (1) PCR preparation after purification of rat liver lysate in chemically modified vessels in the presence of buffer A, (2) PCR preparation after purification of rat liver lysate in chemically modified vessels in the presence of buffer B, (3) PCR preparation after purification of rat liver lysates in chemically modified vessels in the presence of buffer C, (4+5) PCR preparation after purification of rat liver lysates in uncoated vessels in the presence of buffer A, (K⁻) control without gDNA.

The interesting band of the GAPDH gene is in the K⁺ track (control) marked with the arrow. This band is clearly identified also in tracks 1 and 3, less so in track 4; this shows that, in conformity with the invention, enrichment of the double-strand DNA has occurred independent of the buffer used. As demonstrated in tracks 4 and 5, no enrichment occurred when uncoated vessels were used.

1. A method for purification and subsequent determination of double-strand DNA in which the DNA is immobilized on a solid phase during purification, comprising the steps of
coupling a probe to a solid phase, which solid phase is a surface region of a reaction vessel, a chip, or a tip,
bonding the DNA to the probe with a residue having an affinity for DNA,
processing, by PCA or other methods known in the art, the DNA while the nucleic acids remain coupled to the solid phase.

2. A method according to claim 1, wherein the affinity residue of the probe contains zinc finger compounds.

3. A method according to claim 1, wherein the affinity residue of the probe contains DNA binding polyamide compounds.

4. A method according to claim 1, wherein the affinity residue of the probe contains compounds with ionic groups.

5. A method according to claim 1 wherein the further processing of the bound nucleic acids is PCR.

6. A method according to claim 2 wherein the further processing of the bound nucleic acids is PCR.

7. A method according to claim 3 wherein the further processing of the bound nucleic acids is PCR.

8. A method according to claim 1 wherein the further processing is a direct detection of the bound nucleic acids without prior amplification.

9. A method according to claim 2 wherein the further processing is a direct detection of the bound nucleic acids without amplification.

10. A method according to claim 3 wherein the further processing is a direct detection of the bound nucleic acids without amplification.

11. A method according to claim 1, wherein the probe can be bound via a coupling site to the solid-phase carrier by using a streptavidin-biotin system.

12. A method according to claim 2, wherein the probe can be bound via a coupling site to the solid phase carrier by using a streptavidin-biotin system.

13. A method according to claim 3, wherein the probe can be bound via a coupling site to the solid phase carrier by using a streptavidin-biotin system.

14. A method according to claim 4, wherein the probe can be bound via a coupling site to the solid phase carrier by using a streptavidin-biotin system.

15. A method according to claim 5, wherein the probe can be bound via a coupling site to the solid phase carrier by using a streptavidin-biotin system.

16. A method according to claim 6, wherein the probe can be bound via a coupling site to the solid phase carrier by using a streptavidin-biotin system.

17. A method according to claim 7, wherein the probe can be bound via a coupling site to the solid phase carrier by using a streptavidin-biotin system.

18. A method according to claim 8, wherein the probe can be bound via a coupling site to the solid phase carrier by using a streptavidin-biotin system.

19. A method according to claim 9, wherein the probe can be bound via a coupling site to the solid phase carrier by using a streptavidin-biotin system.

20. A method according to claim 10, wherein the probe can be bound via a coupling site to the solid phase carrier by using a streptavidin-biotin system.

21. A reaction vessel or chip which surface is modified by the coupling of a probe with a residue having an affinity for double-strand DNA.
